(19) United States
(12) Patent Application Publication
(10) Pub. No.: US 2024/0293365 A1
(43) Pub. Date: Sep. 5, 2024

(54) METHODS FOR TREATING AND AMELIORATING T CELL RELATED DISEASES

(71) Applicant: The Regents of the University of California, Oakland, CA (US)

(72) Inventors: Yingxiao WANG, San Diego, CA (US); Longwei LIU, San Diego, CA (US); Shaoying LU, San Diego, CA (US)

(21) Appl. No.: 18/575,100

(22) PCT Filed: Jun. 28, 2022
(86) PCT No.: PCT/US2022/035290

§ 371 Date: Dec. 28, 2023

Related U.S. Application Data
(60) Provisional application No. 63/215,866, filed on Jun. 28, 2021.

(51) Int. Cl. A61K 31/404; A61P 37/06

(52) U.S. Cl. CPC A61K 31/404; A61P 37/06

(57) ABSTRACT

In alternative embodiments, provided are methods for treating or ameliorating hyperactive ZAP70 kinase-related diseases or conditions, including any disease or conditions mediated by hyperactive T cells or disease or condition whose pathology is initiated, aggravated or mediated by hyperactive T cells, such as autoimmune disease (including for example, an autoimmune disease requiring allogeneic hematopoietic cell transplantation (HCT) in patients, rheumatoid arthritis (RA), inflammatory bowel disease (IBD), Crohn's disease, type 1 diabetes, multiple sclerosis, uncontrollable bullous pemphigoid, colitis, celiac disease, dermatitis and proteinuria), organ transplant rejection, graft-versus-host disease (GVHD) and/or B cell chronic lymphocytic leukemia (CLL). In alternative embodiments, methods as provided herein comprise administering to an individual in need thereof the FDA-approved cancer drug sunitinib (or SUTENT™), or salts or formulations thereof, for example sunitinib malate.

### RELATED APPLICATIONS

This Patent Convention Treaty (PCT) International Application claims the benefit of priority under 35 U.S.C. § 119(e) of U.S. Provisional Application Ser. No. (USSN) 63/215,866 filed Jun. 28, 2021. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under HL121365, GM125379, GM126016, EB029122, and CA204704, awarded by the National Institutes of Health. The government has certain rights in the invention. The government has certain rights in the invention.

### TECHNICAL FIELD

This invention generally relates to autoimmune diseases and immunology. In alternative embodiments, provided are methods for treating, ameliorating or preventing hyperactive ZAP70 kinase-related disease, infection or conditions, including any disease, infection or conditions mediated by hyperactive T cells or disease or condition whose pathology is initiated, aggravated or mediated by hyperactive T cells, such as autoimmune disease (including for example, an autoimmune disease requiring allogeneic hematopoietic cell transplantation (HCT) in patients, rheumatoid arthritis (RA), lupus erythematosus (LE) or systemic LE, inflammatory bowel disease (IBD), Crohn's disease, type 1 diabetes, multiple sclerosis, uncontrollable bullous pemphigoid, colitis, celiac disease, and proteinuria), organ transplant rejection, graft-versus-host disease (GVHD) and/or B cell chronic lymphocytic leukemia (CLL). In alternative embodiments, methods as provided herein comprise administering to an individual in need thereof the FDA-approved cancer drug sunitinib (or sunitinibum, or SUTENT™), or salts or formulations thereof, for example sunitinib malate.

### BACKGROUND

Genetically-encoded biosensors based on FRET have revolutionized the imaging of molecular signals (for example, protein-protein interactions, protein activations, ion, and small molecule dynamics) in live cells with high spatiotemporal resolution. However, the limited sensitivities of these biosensors have hindered their broader applications in cellular studies and drug screening. At present, optimization of FRET biosensors is rather empirical and labor-intensive, often limited by the availability of accurate protein structures. To address this problem, several methods have been proposed, mainly utilizing evolutionary strategies in bacteria and yeasts. While these methods are very well designed, they generally need additional selection steps to identify the optimized FRET biosensors since results from purified proteins or bacteria/yeasts cannot be translated directly to biosensor responses in mammalian cells. Directed evolution platforms in mammalian cells were established to evolve transcription factors and G protein-coupled receptors (GPCRs), or to optimize the brightness and membrane localization of the voltage reporters utilizing an elegant robotic cell picking system integrated with microscopy. Semi-rational design of relatively small-scale libraries (greater than 100 variants) of FRET biosensors in mammalian cells has also been developed to improve RhoA FRET biosensors. However, there is a lack of method that can systematically engineer and screen relatively large-scale libraries (for example, tens of thousands or larger) of FRET biosensors in mammalian cells for the identification of sensitive biosensors in a high throughput fashion.

FRET biosensors have provided a powerful platform to quantify the dynamics of biochemical¹³⁻¹⁵ and biomechanical¹⁶ signaling in T cells. T Cell-based immunotherapy, for example, CAR-T therapy, has revolutionized cancer treatment. The second-generation design of CARs, containing a CD28 or 4-1BB-derived costimulatory domain at the cytoplasmic tail of CAR, has been widely applied in the clinic¹⁷. Different designs of these CAR molecules, with varying immunoreceptor tyrosine-based activation motifs (ITAMs) at the CAR cytoplasmic tail, have been shown to result in different anti-tumor potencies in vivo. However, the mechanism of ITAM in regulating CAR T cell functions, which is crucial for the design of new CARs, remains unclear. Tyrosine kinases, including Fyn and ZAP70 kinases, serve as key mediators of ITAM and the TCR/CAR cytoplasmic tail. Monitoring these kinases should provide a powerful means to study ITAM functions. At the same time, ZAP70, a key kinase for chronic lymphocytic leukemia (CLL), plays critical roles in T cell signaling and is important in a variety of diseases. For instance, elevated TCR signaling caused by hypermorphic R360P mutation in ZAP70, leads to clinical autoimmune phenotypes characterized by bullous pemphigoid, proteinuria, and colitis. However, due to the low sensitivity of the currently available ZAP70 FRET biosensors and the relatively weak intrinsic activities of ZAP70 kinase, which led to the detection difficulty, methods are not readily available to measure physiologically relevant ZAP70 activities in live cells to study the role of ITAM in regulating TCR/CAR functions.

Despite the crucial roles of ZAP70 kinase in T-cell functions and related diseases, there is no efficient inhibitor targeting ZAP70. HTDS, an effective way to identify kinase inhibitors, has been limited mainly to conventional in vitro enzymatic assay. FRET biosensors can serve as powerful tools for evaluating kinase inhibitors and their related therapeutic drugs in living cells. However, despite the successful integration of FRET biosensors in screening assays for monitoring insulin-receptor activation, and PKA kinase activity, FRET-based biosensors have not been broadly applied for kinase inhibitor screening, mainly due to the relatively small dynamic ranges of FRET biosensors below the greater than 20% dynamic range needed for HTDS assays. Furthermore, these conventional FRET-HTDS assays cannot differentiate inhibitors of upstream signaling molecules from those directly targeting the kinase itself. The heterogeneous levels of kinase activities in individual host cells may impose additional noise and difficulty to the FRET-based screening platforms. Hence, a new FRET-screening design with high-sensitivity biosensors is needed to screen kinase inhibitors in a high-throughput manner.

### SUMMARY

In alternative embodiments, provided methods for treating, ameliorating, preventing, or lessening the amount or severity of symptoms or side effects of: a hyperactive ZAP70 kinase-related disease, infection or condition, or a disease, infection or conditions mediated by a hyperactive T cell or a disease, infection or condition whose pathology is initiated, aggravated or mediated by a hyperactive T cell,

the method comprising:

administering to an individual in need thereof a formulation or product of manufacture comprising sunitinib (or sunitinibum, or SUTENT™), or salts or formulations thereof, optionally sunitinib malate.

In alternative embodiments of methods as provided herein:
the hyperactive ZAP70 kinase-related disease, infection or condition, or disease, infection or condition mediated by a hyperactive T cell, or disease, infection or condition whose pathology is initiated, aggravated or mediated by hyperactive T cells, comprises a disease, infection or condition selected from the group consisting of: an autoimmune disease; an organ transplant rejection; a graft-versus-host disease (GVHD); a B cell chronic lymphocytic leukemia (CLL); a viral infection, and optionally the viral infection comprises: an enterovirus, a Respiratory Syncytial Virus (RSV) infection, a rhinovirus infection, a parainfluenza virus infection, an adenovirus infection, an influenza infection, a herpes infection, a severe acute respiratory syndrome (SARS) infection, or a coronavirus infection, wherein optionally the coronavirus infection is a COVID-19 infection, or an infection by a COVID-19 variant thereof; a virus of the Flaviviridae family or a virus of the genus Hepacivirus or Hepacivirus C virus or hepatitis C; a skin condition, wherein optionally the skin condition is vitiligo, psoriasis, pemphigus, pemphigoid or atopic dermatitis,

and optionally the administration prevents, lessens or ameliorates a myalgia, a rhabdomyolysis and/or an encephalopathy caused by the hyperactive ZAP70 kinase-related disease, infection or condition, or disease, infection or condition;
the autoimmune disease is or comprises: an allogeneic hematopoietic cell transplantation (HCT) in patients, rheumatoid arthritis (RA), lupus erythematosus (LE) or systemic LE, inflammatory bowel disease (IBD), Crohn's disease, Grave's disease or Hashimoto's thyroiditis, scleroderma, Guillain-Barre syndrome, type 1 diabetes, multiple sclerosis, uncontrollable bullous pemphigoid, psoriasis, colitis, celiac disease, dermatitis and proteinuria;

the formulation or product of manufacture is administered by inhalation, intravenously (IV), intradermally, intrathecally, sub-or intra-dermally, topically or intramuscularly (IM), and optionally the formulation is formulated for administration in vivo; or as a pharmaceutical formulation or composition, or for enteral or parenteral administration, or as a tablet, pill, capsule, lozenge, gel, hydrogel, geltab, liquid, lotion, aerosol, patch, spray, or implant, and optionally the formulation is formulated as a liposome, a nanoparticle or a nanolipoparticle; and/or
the sunitinib (or SUTENT™), or salts or formulations thereof, optionally sunitinib malate is administered using, administering or implanting: a kit, an implant, a pump, a device, a subcutaneous infusion device, a continuous subcutaneous infusion device, an infusion pen, a needle, a reservoir, an ampoules, a vial, a syringe, a cartridge, a pen, a disposable pen or jet injector, a prefilled pen or a syringe or a cartridge, a cartridge or a disposable pen or jet injector, a two chambered or multi-chambered pump.

In alternative embodiments, provided are uses of a formulation or product of manufacture comprising sunitinib (or SUTENT™), or salts or formulations thereof, optionally sunitinib malate, for treating or ameliorating a hyperactive ZAP70 kinase-related disease, infection or condition, or a disease, infection or conditions mediated by a hyperactive T cell or a disease or condition whose pathology is initiated, aggravated or mediated by a hyperactive T cell.

In alternative embodiments, provided are formulations or products of manufacture comprising sunitinib (or SUTENT™), or salts or formulations thereof, optionally sunitinib malate, for use in treating or ameliorating a hyperactive ZAP70 kinase-related disease, infection or condition, or a disease, infection or conditions mediated by a hyperactive T cell or a disease or condition whose pathology is initiated, aggravated or mediated by a hyperactive T cell.

In alternative embodiments, provided are methods for inhibiting the activity of a cell comprising a ZAP70 kinase in vivo, in vitro or ex vivo, comprising contacting the cell with a formulation or product of manufacture comprising sunitinib (or SUTENT™), or salts or formulations thereof, optionally sunitinib malate. In alternative embodiments, the cell is a lymphocyte, the lymphocyte is a T cell, a B cell or a natural killer (NK) cell, or, the cell is a hyperactive T cell that mediates or initiates or aggravates a disease, infection or condition. In alternative embodiments, the disease, infection or condition is selected from the group consisting of: an autoimmune disease; organ transplant rejection; graft-versus-host disease (GVHD); a B cell chronic lymphocytic leukemia (CLL); a viral infection, and optionally the viral infection comprises: an enterovirus, a Respiratory Syncytial Virus (RSV) infection, a rhinovirus infection, a parainfluenza virus infection, an adenovirus infection, an influenza infection, a herpes infection, a severe acute respiratory syndrome (SARS) infection, or a coronavirus infection, wherein optionally the coronavirus infection is a COVID-19 infection, or an infection by a COVID-19 variant thereof, ol a virus of the Flaviviridae family or a virus of the genus Hepacivirus or Hepacivirus C virus or hepatitis C; and/or a skin condition, wherein optionally the skin condition is vitiligo, psoriasis, pemphigus, pemphigoid or atopic dermatitis. In alternative embodiments, the autoimmune disease is or comprises: an allogeneic hematopoietic cell transplantation (HCT) in patients, rheumatoid arthritis (RA), lupus erythematosus (LE) or systemic LE, inflammatory bowel disease (IBD), Crohn's disease, Grave's disease or Hashimoto's thyroiditis, scleroderma, vasculitis, Guillain-Barre syndrome, type 1 diabetes, multiple sclerosis, uncontrollable bullous pemphigoid, colitis, and proteinuria.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-D illustrate an exemplary saFRET assay specific to ZAP70 kinase:

FIG. 1A illustrates a schematic of a conventional FRET assay in T cells;

FIG. 1B illustrates data showing that Src, Fyn, Lck, and Syk kinase inhibitors can reduce the FRET change of conventional biosensors after CD3/CD28 antibody stimulation;

FIG. 1C illustrates a schematic of an exemplary saFRET assay in HEK293 cells, where the FRET change is mediated by the kinase domain;

FIG. 1D illustrates data showing that inhibitors of Src, Fyn, Lck, and Syk kinases do not cause a significant FRET change of the ZAP70 saFRET biosensor,

as further described in Example 1, below.

FIG. 2A-E illustrate the identification of sunitinib as a ZAP70 kinase inhibitor from a library:

FIG. 2A illustrates a schematic of an exemplary high throughput drug screening platform, with cells cultured in 96-well glass-bottom plate treated either with DMSO or inhibitors from the kinase inhibitor library;

FIG. 2B illustrates FRET-Ratio images of the cells with different kinase inhibitors (PHA-665752; BIBF 1120; staurosporine; sunitinib; FRAX 597; AZD 1208; AZD 7762), and DMSO as a negative control;

FIG. 2C graphically illustrates a summary of screening results, where some of the inhibitors have shown high efficiency in inhibiting ZAP70 kinase, and the highlighted inhibitors represent the inhibitors targeting ZAP70 upstream signaling molecules (dasatinib, Src kinase inhibitor; PP2, Lck/Fyn kinase inhibitor; R406, Syk inhibitor);

FIG. 2D graphically illustrates the top 10 selected inhibitors: PHA-665752; BIBF 1120; staurosporine; sunitinib; FRAX 597; AZD 1208; ZM 447439; AZD 7762; Tie2 kinase inhibitor and NVP-TAE226; and

FIG. 2E graphically illustrates counter screening using a mutant biosensor with a kinase-dead domain to subtract the noise engendered from non-specific fluorescence, the scatter plot illustrates the FRET ratio changes in the positive and negative screenings using the saFRET biosensor fused with an active kinase or a kinase-dead domain, respectively,

as further described in Example 1, below.

FIG. 3A-F illustrate verification of sunitinib as a ZAP70 inhibitor in a ZAP70-R360P mutant induced T cell activation model:

FIG. 3A illustrates an exemplary experimental scheme and timeline of P116 cells reconstituted with ZAP70, where full-length ZAP70-WT or R360P were expressed with YPet via a cleavable P2A linker;

FIG. 3B graphically illustrates CD69 expression in P116 cells with or without the expression of ZAP70 (WT) and its mutant (R360P);

FIG. 3C illustrates images of pZAP70-Y493(active ZAP70 kinase marker) in P116-ZAP70 R360P cells with sunitinib pre-treatment;

FIG. 3D graphically illustrates quantification of pZAP70 (Y493) intensity of P116-ZAP70 R360P cells with different inhibitor pre-treatments;

FIG. 3E illustrates images of pLAT (Y191) in P116-ZAP70 R360P cells with sunitinib pre-treatment; and

FIG. 3F graphically illustrates quantification of pLAT (Y191) intensity of P116-ZAP70 R360P cells with different inhibitor pre-treatments,

as further described in Example 1, below.

FIG. 4A-E illustrate verification of sunitinib as ZAP70 inhibitor in a general T cell activation model:

FIG. 4A schematically illustrates an experimental scheme and timeline for experiments in FIG. 4B-D, where Jurkat T cells were pretreated with inhibitors for 30 min before anti-TCR stimulation by anti-CD3/CD28 antibodies for 5 min;

FIG. 4B illustrates images of pZAP70-Y493 (active ZAP70 kinase marker) in T cells with sunitinib pre-treatment;

FIG. 4C graphically illustrates quantification of pZAP70 (Y493) intensity of single cells in different groups;

FIG. 4D illustrates images of pLAT (Y191) in T cells with sunitinib pre-treatment; and

FIG. 4E graphically illustrates quantification of pLAT (Y191) intensity of single cells in different groups,

as further described in Example 1, below.

FIG. 5A-C illustrate that sunitinib was effective in inhibiting the hyperactive T cells:

FIG. 5A illustrates an exemplary experimental scheme and timeline for CD69 staining experiment;

FIG. 5A illustrates a flow-cytometry analysis of CD69 expression in P116-ZAP70-R360P cells after anti-TCR stimulation, with or without sunitinib pre-treatment;

FIG. 5C illustrates a flow-cytometry analysis of CD69 expression in T cells after anti-TCR stimulation, with or without sunitinib pre-treatment,

as further described in Example 1, below.

Like reference symbols in the various drawings indicate like elements.

### DETAILED DESCRIPTION

In alternative embodiments, provided are methods for treating or ameliorating hyperactive ZAP70 kinase-related diseases, infections or conditions, including any disease, infection or condition mediated by hyperactive T cells or any disease, infection or condition whose pathology is initiated, aggravated or mediated by a hyperactive T cell, such as for example an autoimmune disease (including for example, an autoimmune disease requiring allogeneic hematopoietic cell transplantation (HCT) in patients, rheumatoid arthritis (RA), lupus erythematosus (LE) or systemic LE, inflammatory bowel disease (IBD), Crohn's disease, Grave's disease or Hashimoto's thyroiditis, scleroderma, vasculitis, Guillain-Barre syndrome, type 1 diabetes, multiple sclerosis, uncontrollable bullous pemphigoid, colitis, and proteinuria), organ transplant rejection, graft-versus-host disease (GVHD), a B cell chronic lymphocytic leukemia (CLL), a viral infection, wherein optionally the viral infection comprises: an enterovirus, a Respiratory Syncytial Virus (RSV) infection, a rhinovirus infection, a parainfluenza virus infection, an adenovirus infection, an influenza infection, a herpes infection, or a coronavirus infection, wherein optionally the coronavirus infection is a COVID-19 infection, or an infection by a COVID-19 variant thereof, or virus of the Flaviviridae family or a virus of the genus Hepacivirus or Hepacivirus C virus or hepatitis C; or a skin condition, wherein optionally the skin condition is vitiligo, psoriasis, pemphigus, pemphigoid or atopic dermatitis. In alternative embodiments, methods as provided herein comprise administering to an individual in need thereof the FDA-approved cancer drug sunitinib (or SUTENT™), or salts or formulations thereof, for example sunitinib malate.

In alternative embodiments, provided are broader applications for Förster Resonance Energy Transfer (FRET) biosensors that expand their sensitivities. Here, we describe a systematic approach coupling high-throughput FRET sorting and next-generation sequencing (FRET-Seq) to identify sensitive biosensors from large-scale libraries directly in mammalian cells, utilizing the design of self-activating FRET (saFRET) biosensor. The resulting biosensors of Fyn and ZAP70 kinases exhibit high-performance and enabled the dynamic imaging of T cell activation mediated by T cell receptor (TCR) and chimeric antigen receptor (CAR), revealing a highly organized ZAP70 subcellular activity pattern upon TCR but not CAR engagement. The ZAP70-biosensor also allowed elucidating the role of immunoreceptor tyrosine-based activation motif (ITAM) in affecting ZAP70 activation to regulate CAR functions. A saFRET biosensor-based high-throughput drug screening (saFRET-HTDS) assay further allowed the identification of an FDA-approved cancer drug, sunitinib (or SUTENT™), that can be repurposed to inhibit ZAP70 activity and autoimmune-disease-related T-cell activation.

### Formulations and Pharmaceutical Compositions

In alternative embodiments, provided are compounds and compositions, including formulations and pharmaceutical compositions, for use in in vivo, in vitro or ex vivo methods for inhibiting the activity of a T cell comprising a ZAP70 kinase, including treating or ameliorating hyperactive ZAP70 kinase-related diseases, infection or conditions, including any disease, infection or conditions mediated by hyperactive T cells or disease, infection or condition whose pathology is initiated, aggravated or mediated by hyperactive T cells.

In alternative embodiments, the pharmaceutical compositions used in methods as provided herein can be administered parenterally, topically, orally or by local administration, such as by aerosol or transdermally. In alternative embodiments, pharmaceutical compositions used in methods as provided herein can be prepared in various forms, such as granules, tablets, pills, capsules, suspensions, taken orally, suppositories and salves, lotions and the like. Pharmaceutical formulations used in methods as provided herein may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, lozenges, gels, geltabs, on patches, in implants, etc. In practicing embodiments as provided herein, the pharmaceutical compounds can be delivered by transdermally, by a topical route, formulated as applicator sticks, solutions, suspensions, emulsions, gels, creams, ointments, pastes, jellies, paints, powders, and aerosols. Oral carriers can be elixirs, syrups, capsules, tablets, pills, geltabs and the like.

In alternative embodiments, pharmaceutically acceptable salts of compounds used in methods as provided herein include pharmaceutically acceptable non-toxic bases or acids including inorganic or organic bases and inorganic or organic acids. In alternative embodiments, salts are derived from inorganic bases such as aluminum, ammonium, calcium, copper, ferric, ferrous, lithium, magnesium, manganic salts, manganese, potassium, sodium, zinc, and the like; or, salts can be in a solid form, or in a crystal structure, or the form of hydrates. In alternative embodiments, salts are pharmaceutically acceptable organic non-toxic bases including salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. In alternative embodiments, for example, if a compound provided herein is basic, salts are prepared from pharmaceutically acceptable non-toxic acids, including inorganic and organic acids. Such acids include acetic, benzenesulfonic, benzoic, camphorsulfonic, carbonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In alternative embodiments, pharmaceutically acceptable salts include hemisalts of non-toxic acids or bases, or hemihydrates.

In alternative embodiments, compounds and compositions used to practice methods as provided herein, are delivered orally, for example, as pharmaceutical formulations for oral administration, and can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients can be carbohydrate or protein fillers, for example, sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins, for example, gelatin and collagen. Disintegrating or solubilizing agents may be added, such as the cross-linked polyvinyl pyrrolidone, agar, alginic acid, or a salt thereof, such as sodium alginate.

In alternative embodiments, liquid carriers are used to manufacture or formulate compounds as provided herein, or a composition used to practice the methods as provided herein, including carriers for preparing solutions, suspensions, emulsions, syrups, elixirs and pressurized compounds. The active ingredient (for example, a composition as provided herein) can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, solid carriers are used to manufacture or formulate compounds or a composition used to practice the methods as provided herein, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (for example, povidone, gelatin, hydroxypropylmethyl cellulose), lubricant, inert diluent, preservative, disintegrant (for example, sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, concentrations of therapeutically active compound in a formulation can be from between about 0.1% to about 100% by weight.

In alternative embodiments, therapeutic formulations are prepared by any method well known in the art, for example, as described by Brunton et al., eds., Goodman and Gilman's: The Pharmacological Bases of Therapeutics, 12th ed., McGraw-Hill, 2011; Remington: The Science and Practice of Pharmacy, Mack Publishing Co., 20th ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

In alternative embodiments, therapeutic formulations are delivered by any effective means appropriated for a particular treatment. For example, depending on the specific antitumor agent to be administered, the suitable means include oral, rectal, vaginal, nasal, pulmonary administration, or parenteral (including subcutaneous, intramuscular, intravenous and intradermal) infusion into the bloodstream. For parenteral administration, antitumor agents as provided herein may be formulated in a variety of ways. Aqueous solutions of the modulators can be encapsulated in polymeric beads, liposomes, nanoparticles or other injectable depot formulations known to those of skill in the art. In alternative embodiments, compounds and compositions as provided herein, or a compound used to practice methods as provided herein, are administered encapsulated in liposomes (see below). In alternative embodiments, depending upon solubility, compositions are present both in an aqueous layer and in a lipidic layer, for example, a liposomic suspension. In alternative embodiments, a hydrophobic layer comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The pharmaceutical compositions or formulation or product of manufacture comprising sunitinib (or SUTENT™), or salts or formulations thereof, optionally sunitinib malate, can be formulated in any way and can be administered in a variety of unit dosage forms depending upon the condition or disease or infection and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”). For example, in alternative embodiments, compounds and compositions as provided herein, or a compound used to practice methods as provided herein, are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice embodiments as provided herein can be in forms such as tablets, pills, powders, capsules, liquids, gels, syrups, slurries, suspensions, etc., suitable for in vivo, in vitro or ex vivo applications.

In practicing embodiments as provided herein, the formulations used to practice methods as provided herein can comprise a solution of compositions disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose, any fixed oil can be employed including synthetic mono-or diglycerides, or fatty acids such as oleic acid. In one embodiment, solutions and formulations used to practice embodiments as provided herein are sterile and can be manufactured to be generally free of undesirable matter. In one embodiment, these solutions and formulations are sterilized by conventional, well known sterilization techniques.

The solutions and formulations used to practice methods as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compounds and compositions used to practice methods as provided herein, can be delivered by the use of liposomes. In alternative embodiments, by using liposomes, particularly where the liposome surface carries ligands specific for target cells or organs, or are otherwise preferentially directed to a specific tissue or organ type, one can focus the delivery of the active agent into a target cells in an in vivo, in vitro or ex vivo application.

The compounds and compositions used to practice methods as provided herein, can be directly administered, for example, under sterile conditions, to an individual (for example, a patient) to be treated. The modulators can be administered alone or as the active ingredient of a pharmaceutical composition. Compositions and formulations as provided herein can be combined with or used in association with other therapeutic agents. For example, an individual may be treated concurrently with conventional therapeutic agents.

### Nanoparticles, Nanolipoparticles and Liposomes

Provided are particles, microparticles, nanoparticles, nanolipoparticles, vesicles and liposomal membranes comprising compounds and compositions used to practice the methods and embodiments as provided herein. Provided are multilayered liposomes comprising compounds used to practice embodiments as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice embodiments as provided herein.

Liposomes can be made using any method, for example, as described in Park, et al., U.S. Pat. Pub. No. 20070042031, including the method of producing a liposome by encapsulating an active agent (for example, compounds and compositions as provided herein, or a compound used to practice methods as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, for example, for targeting delivery of a compound as provided herein, or a compound used to practice methods as provided herein, to a desired cell type or organ, for example, brain, as described for example, in U.S. Pat. Pub. No. 20070110798.

Provided are nanoparticles comprising compounds as provided herein, for example, used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent used to practice embodiments as provided herein, or a fat-solubilized water-soluble active agent to act with a bivalent or trivalent metal salt.

In one embodiment, solid lipid suspensions can be used to formulate and to deliver compositions used to practice embodiments as provided herein to mammalian cells in vivo, in vitro or ex vivo, as described, for example, in U.S. Pat. Pub. No. 20050136121.

### Delivery Vehicles

In alternative embodiments, any delivery vehicle can be used to practice the methods as provided herein, for example, to deliver a compound used to practice methods as provided herein, for example, to deliver a formulation or product of manufacture comprising sunitinib (or SUTENT™), or salts or formulations thereof, optionally sunitinib malate, to mammalian cells, for example, in vivo, in vitro or ex vivo. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate compounds and compositions as provided herein, or a compound used to practice embodiments as provided herein, for example as described, for example, in U.S. Pat. Pub. No. 20040151766.

In one embodiment, compounds and compositions used to practice methods as provided herein, can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, for example, using any electroporation system as described for example in U.S. Pat. Nos. 7,109,034; 6,261,815; 5,874,268.

### Dosaging

The pharmaceutical compositions and formulations used to practice methods as provided herein to deliver a formulation or product of manufacture comprising sunitinib (or SUTENT™), or salts or formulations thereof, optionally sunitinib malate, can be administered for prophylactic and/or therapeutic treatments. In therapeutic applications, compositions, pharmaceutical compositions or formulations are administered to a subject or individual, for example, a human in need thereof, in an amount of the agent sufficient to be therapeutically effective, for example, to cure, alleviate or partially arrest the clinical manifestations and/or its complications, or for example, for treating, ameliorating, preventing, reversing or slowing the progression of: a cancer, a tumor, a metastasis or a dysplastic or a dysfunctional cell condition responsive to inhibition of a kinase enzyme, or for example, for treating, ameliorating, preventing or reversing, slowing the progression of, or decreasing the severity of: an autoimmune disease or condition, an inflammatory disease or condition, an inherited or genetic disease or condition, a neurodegenerative disease or condition, or an infection responsive to inhibition of a kinase enzyme (a “therapeutically effective amount”).

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease, infection or condition, the severity of the disease, infection or condition, the general state of the patient's health, the patient's physical status, age and the like. Dosage levels (of, for example, sunitinib (or SUTENT™), or salts or formulations thereof, optionally sunitinib malate) may range from about 0.01 mg per kilogram to about 100 mg per kilogram of body weight. In calculating the dosage regimen for a patient, the mode of administration also is taken into consideration. In alternative embodiments, sunitinib (or SUTENT™), or salts or formulations thereof, optionally sunitinib malate) is administered at dosages of 5, 10, 12.5, 15, 20, 25, 30, 35, 37.5, 40, 45, 50, 55, 60, 65, 70, 75, 80, 85, 90 or 100 mg, or between 5 and 150 mg per unit dose, or 5, 10, 12.5, 15, 20, 25, 30, 35, 37.5, 40, 45, 50, 55, 60, 65, 70, 75, 80, 85, 90 or 100 mg, or between 5 and 150 mg, per day. In alternative embodiments, sunitinib (or SUTENT™), or salts or formulations thereof, optionally sunitinib malate) is administered at these dosages for between one week and one month, optionally every day or every other day. In alternative embodiments, sunitinib (or SUTENT™), or salts or formulations thereof, optionally sunitinib malate) is administered at these dosages for one to two weeks, followed by a one to two week break, followed again by a one to two week treatment regimen (where drug is administered every day, or every other day). In alternative embodiments, sunitinib (or SUTENT™), or salts or formulations thereof, optionally sunitinib malate) is administered orally, for example, in capsule, tablet or geltab form, wherein optionally the unit dosage is 12.5, 25 or 50 mg.

The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24: 103-108; the latest Remington's, supra). The state of the art allows the clinician to determine the dosage regimen for each individual patient, active agent and disease, infection or condition treated. Guidelines provided for similar compositions used as pharmaceuticals can be used as guidance to determine the dosage regiment, i.e., dose schedule and dosage levels, administered practicing the methods as provided herein are correct and appropriate.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1

This example demonstrates that methods as provided herein using sunitinib (or SUTENT™), or salts or formulations thereof, optionally sunitinib malate, are effective and can be used for treating or ameliorating a hyperactive ZAP70 kinase-related disease, infection or condition, or a disease, infection or conditions mediated by a hyperactive T cell or a disease, infection or condition whose pathology is initiated, aggravated or mediated by a hyperactive T cell.

Apply ZAP70 saFRET Assay to Find ZAP0 Inhibitors of High Specificity.

The saFRET biosensor design provides a platform for biosensor-based high-throughput drug screening (HTDS) in living cells. The carry-on-kinase-domain should bypass the need to maintain endogenous regulation of the target kinase, minimize the heterogeneity and noise of endogenous kinase activation, avoid identifying pathway regulators instead of on-target hits, and provide flexibility of choosing suitable cell systems that maintain live-cell contexts but offer experimental ease.

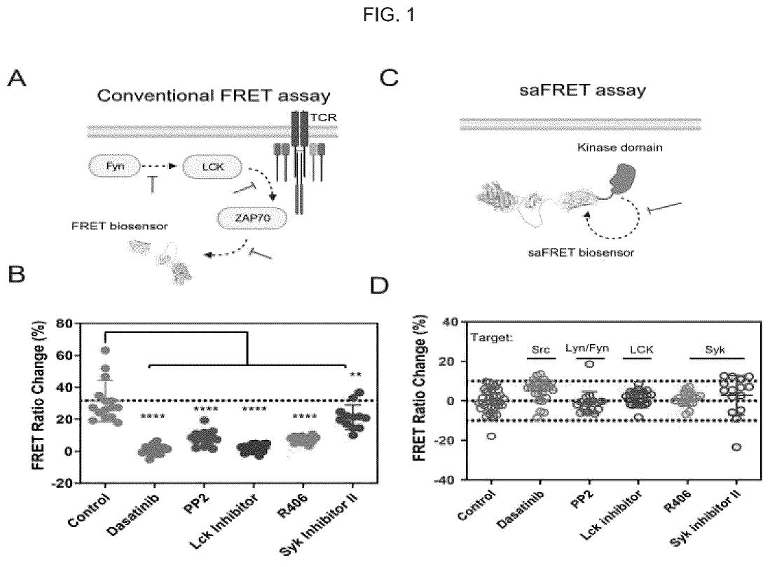

Compared to conventional assays, saFRET-HTDS enables the screening of inhibitors directly targeting ZAP70 kinase with high specificity, avoiding the false positive selection of inhibitors targeting upstream molecules instead of ZAP70 itself in host cells. Indeed, our results showed that the hits using the conventional FRET biosensor assay include inhibitors that target ZAP70 upstream signaling molecules (FIG. 1A-B), while the saFRET assay showed no response to these off-target inhibitors (FIG. 1C-D). These results demonstrate the higher specificity of our saFRET screening assays over the conventional FRET assays; thus, the positive hits should high specificity to ZAP70 kinase.

Sunitinib was Identified to Inhibit the ZAP70 Kinase Activity from an Inhibitor Library Significantly

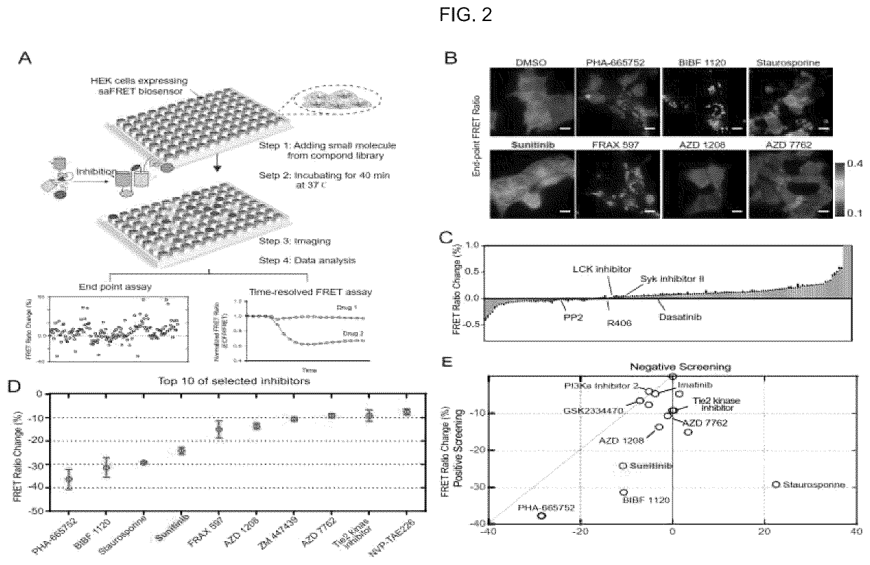

We first screened a 96-member kinase inhibitor library to identify efficient inhibitors of ZAP70 kinase. During the screening, library inhibitors at 10 μM were used as the screening dosage to identify the inhibitors more potent than TAK-659, which was relatively ineffective in suppressing ZAP70 activity at 10 μM. After 40 minutes of incubation with inhibitors, the cells were imaged, and the FRET ratio changes of individual inhibitors compared to the solvent control were calculated to identify promising candidates after this primary endpoint screening (FIG. 2). We further exploited a control biosensor with a kinase-dead domain (saFRETkd) for counter screening to eliminate false-positive hits due to auto-fluorescence or other non-specific effects (1).

Among the candidates identified, sunitinib showed high efficiency in inhibiting the ZAP70 kinase domain-mediated activation saFRET biosensor (FIG. 2B) and ranked 4th (FIG. 2C-D) saFRET screening results. The counter screening illustrated that sunitinib has fluorescence may influence the FRET signal of saFRETkd, but this effect is not significant compared to the positive screening (FIG. 2E). In summary, sunitinib was identified to inhibit the ZAP70 kinase activity from an inhibitor library significantly.

### Sunitinib was Found to Inhibit ZAP70 Kinases in a ZAP70-R360P Mutant Induced T Cell Activation Model.

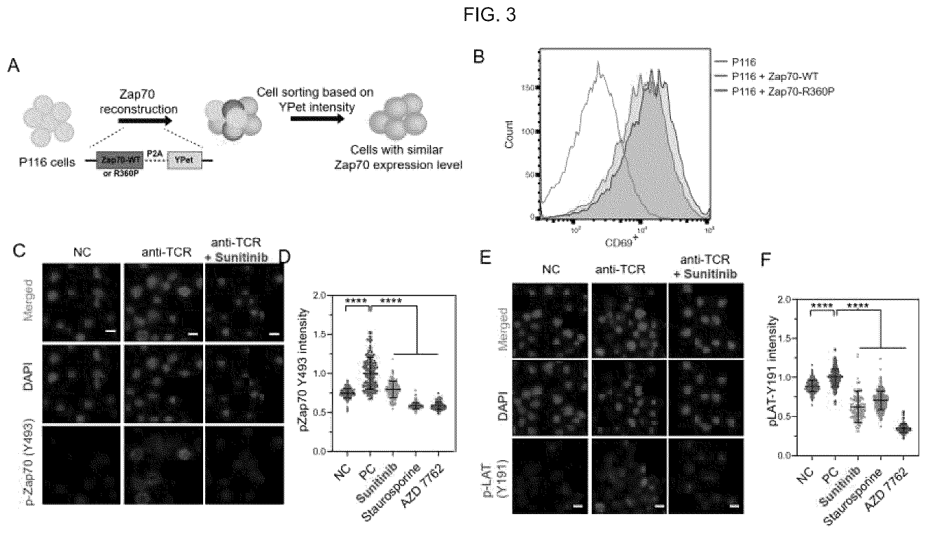

Next, we tested whether the sunitinib could target the ZAP70 kinase in hyperactive T cells. We used a disease model where T cell activation is mediated by ZAP70-R360P mutation, the main cause of a severe human autoimmune syndrome (2). We introduced wild-type ZAP70 or the R360P-mutant of ZAP70 into ZAP70-deficient P116 T cells (FIG. 3A), in which ZAP70-R360P led to an enhanced T cell auto-activation compared to the wild-type ZAP70 (2) (FIG. 3B). Using this model, we further measured the activation of ZAP70 and the phosphorylation of LAT, a downstream substrate of ZAP70 kinase, in the R360P mutation-mediated hyperactive T cell model. Significant reductions of phosphorylated ZAP70 (FIG. 3C-D) and its downstream substrate LAT(Y191) (FIG. 3E-F) were observed after treatments with sunitinib, verifying the efficacy of the biosensor screening results. These experiments demonstrated that sunitinib could be used as a drug to manage the activation of hyperactive ZAP70-R360P kinase.

### Sunitinib was Found to Inhibit ZAP70 Kinases in a General Hyperactive T Cell Model.

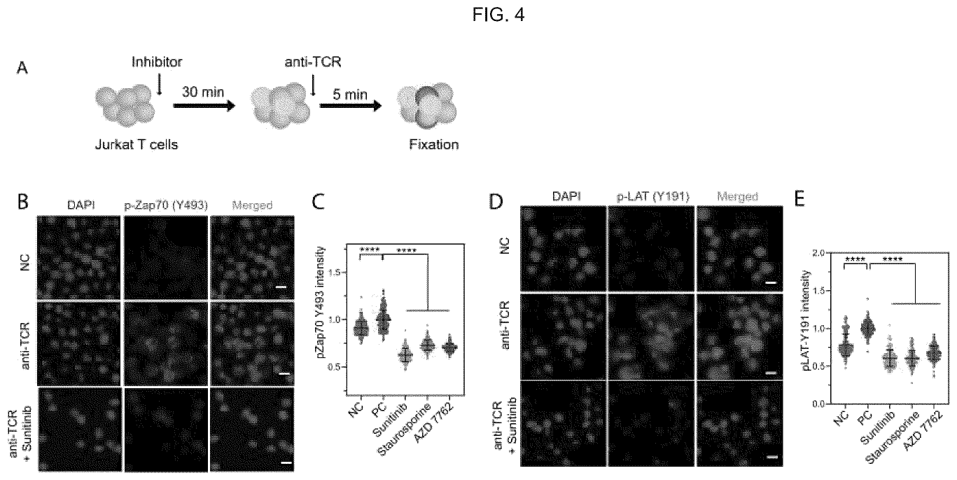

We further measured whether sunitinib could be used as a general approach to inhibit the activation of ZAP70 kinase and the subsequent hyperactive T cell phenotype. Thus, we further tested the effectiveness of sunitinib in a general hyperactive T cell model in which the T cells were activated by an anti-TCR (C305) antibody. Significant reductions of phosphorylated ZAP70 and its downstream substrate LAT(Y191) were also observed after treatments with sunitinib, verifying the efficacy of the biosensor screening results (FIG. 4). These experiments demonstrated that sunitinib could be used as a drug potentially for any hyperactive ZAP70 related diseases.

Sunitinib was Effective in Inhibiting the Hyperactive T cells.

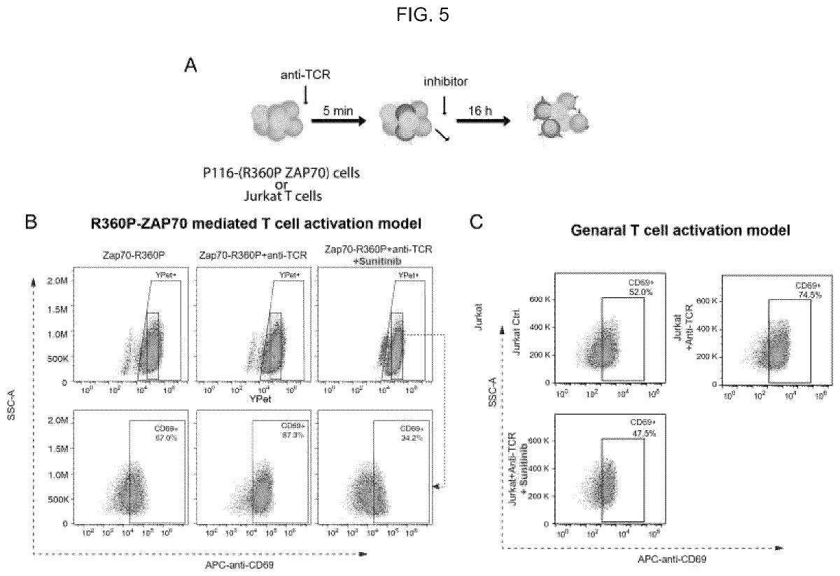

Finally, we tested whether the sunitinib could be used to inhibit the activation of T cells in both the ZAP70-R360P mutant induced T cell activation model and the general T cell activation model (FIG. 5A). CD69, a marker for activated T cells, was measured in hyperactivated T cells threated with or without sunitinib treatment. A significant increase of CD69 expression was found after these T cells were activated by anti-TCR. This hyperactivation of T cells could be abolished after sunitinib treatment (FIG. 5B-c), demonstrating the efficiency of sunitinib in migrating hyperactive T cells. Hence, sunitinib identified by our saFRET screening may be applicable to mitigate abnormal ZAP70 activation or ZAP70R360P related autoimmune diseases.

Since sunitinib is an FDA-approved drug whose safety has been demonstrated in human subjects, we expect that sunitinib has high therapeutic potential to be repurposed in treating hyperactive ZAP70-related autoimmune diseases (for example, R360P mutation has been shown to cause autoimmune disease requiring allogeneic hematopoietic cell transplantation in patient).

### FIGURE AND FIGURE LEGENDS

FIG. 1. The saFRET Assay is Specific to ZAP70 Kinase.

A, Schematics of conventional FRET assay in T cells. The FRET change could be affected by inhibitors targeting ZAP70 kinase and its upstream molecules.

B, The Src, Fyn, Lck, and Syk kinase inhibitors could reduce the FRET change of conventional biosensors after CD3/CD28 antibody stimulation significantly. The inhibitor groups are pretreated with corresponding inhibitors as indicated for 30 min before antibody stimulation. Control indicates the DMSO pretreated group. (n>12 in each group, One-way ANOVA, ****P<0.0001 and **P=0.0013).

C, Schematics of saFRET assay in HEK293 cells. The FRET change is mediated by the kinase domain.

D, Inhibitors of Src, Fyn, Lck, and Syk kinases could not cause a significant FRET change of the ZAP70 saFRET biosensor. Control indicates the DMSO treated group. (n>15 in each group and data were tested by One-way ANOVA).

FIG. 2. The Identification of sunitinib as a ZAP70 Kinase Inhibitor from a Library.

A, Schematics of the high throughput drug screening platform. First, the cells cultured in 96-well glass-bottom plate were treated either with DMSO or inhibitors from the kinase inhibitor library. After 40 minutes of incubation, the cells were imaged, and the FRET ratio change compared to the control cell was calculated. This platform can also allow dynamic tracking of the FRET ratio change after inhibitor treatment in single cells.

B, FRET-Ratio images of the cells with different inhibitors. Scale bars, 10 μm.

C, summary of screening results. Some of the inhibitors have shown high efficiency in inhibiting ZAP70 kinase. The highlighted inhibitors represent the inhibitors targeting ZAP70 upstream signaling molecules. Dasatinib, Src kinase inhibitor; PP2, Lck/Fyn kinase inhibitor; R406, Syk inhibitor.

D, Top 10 selected inhibitors (n≤25 for each group). Error bars, mean±SD.

E, Counter screening using a mutant biosensor with a kinase-dead domain to subtract the noise engendered from non-specific fluorescence. The Scatter plot illustrates the FRET ratio changes in the positive and negative screenings using the saFRET biosensor fused with an active kinase or a kinase-dead domain, respectively.

FIG. 3. Verification of sunitinib as ZAP70 Inhibitor in a ZAP70-R360P Mutant Induced T Cell Activation Model.

A, Experimental scheme and timeline of P116 cells reconstituted with ZAP70. Full-length ZAP70-WT or R360P were expressed with YPet via a cleavable P2A linker. P116 cells with similar ZAP70-WT or ZAP70-R360P expressions were sorted and isolated for further analysis based on YPet intensity.

B, CD69 expression in P116 cells with or without the expression of ZAP70 (WT) and its mutant (R360P).

C, Images of pZAP70-Y493(active ZAP70 kinase marker) in P116-ZAP70 R360P cells with sunitinib pre-treatment. Scale bars, 10 μm.

D. Quantification of pZAP70 (Y493) intensity of P116-ZAP70 R360P cells with different inhibitor pre-treatments. (n>100 for each group, One-way ANOVA, ****P<0.0001). Error bars, mean±SD.

E, Images of pLAT (Y191) in P116-ZAP70 R360P cells with sunitinib pre-treatment. Scale bars, 10 μm.

F, Quantification of pLAT (Y191) intensity of P116-ZAP70 R360P cells with different inhibitor pre-treatments. (n>150 for each group, One-way ANOVA, **** P<0.0001). Error bars, mean±SD.

FIG. 4. Verification of sunitinib as ZAP70 Inhibitor in a General T Cell Activation Model.

A, Experimental scheme and timeline for experiments in b-d. Jurkat T cells were pretreated with inhibitors for 30 min before anti-TCR stimulation by anti-CD3/CD28 antibodies for 5 min.

B, Images of pZAP70-Y493(active ZAP70 kinase marker) in T cells with sunitinib pre-treatment. Scale bars, 10 μm.

C. Quantification of pZAP70-(Y493) intensity of single cells in different groups. (n>200 for each group, One-way ANOVA, ****P<0.0001). Error bars, mean±SD.

D, Images of pLAT (Y191) in T cells with sunitinib pre-treatment. Scale bars, 10 μm. E, Quantification of pLAT (Y191) intensity of single cells in different groups. (n>150 for each group, One-way ANOVA, ****P<0.0001). NC represents Jurkat T cells without any treatment, PC represents Jurkat T cells stimulated with anti-TCR only. Data were normalized to the PC group. Error bars, mean±SD.

### FIG. 5. Sunitinib was Effective in Inhibiting the Hyperactive T Cells.

A, Experimental scheme and timeline for CD69 staining experiment.

B, Flow-cytometry analysis of CD69 expression in P116-ZAP70-R360P cells after anti-TCR stimulation, with or without sunitinib pre-treatment. ZAP70-R360P expression levels in P116 cells were indicated by YPet intensity.

C.Flow-cytometry analysis of CD69 expression in T cells after anti-TCR stimulation, with or without sunitinib pre-treatment.

### REFERENCES

1. Simeonov A, Davis M I. Interference with Fluorescence and Absorbance. In: Markossian S, et al. editors. Assay Guidance Manual. Bethesda (MD): Eli Lilly & Company and the National Center for Advancing Translational Sciences; 2004.

2. Chan A Y, et al. A novel human autoimmune syndrome caused by combined hypomorphic and activating mutations in ZAP-70. J Exp Med. 2016; 213(2):155-65. Epub 2016/01/18. doi: 10.1084/jem.20150888. PubMed PMID: 26783323.

A number of embodiments of the invention have been described. Nevertheless, it can be understood that various modifications may be made without departing from the spirit and scope of the invention. Accordingly, other embodiments are within the scope of the following claims.

1. A method for treating or ameliorating a hyperactive ZAP70 kinase-related disease or condition, or a disease or conditions mediated by a hyperactive T cell or a disease or condition whose pathology is initiated, aggravated or mediated by a hyperactive T cell,
the method comprising:
administering to an individual in need thereof a formulation or product of manufacture comprising sunitinib, or salts or formulations thereof.

2. The method of claim 1, wherein the hyperactive ZAP70 kinase-related disease or condition, or disease or condition mediated by a hyperactive T cell, or disease or condition whose pathology is initiated, aggravated or mediated by hyperactive T cells, comprises a disease or condition selected from the group consisting of: an autoimmune disease, organ transplant rejection, graft-versus-host disease (GVHD) and B cell chronic lymphocytic leukemia (CLL).

3. The method of claim 2, wherein the autoimmune disease is or comprises: an allogeneic hematopoietic cell transplantation (HCT) in patients, rheumatoid arthritis (RA), inflammatory bowel disease (IBD), Crohn's disease, type 1 diabetes, multiple sclerosis, uncontrollable bullous pemphigoid, colitis, and proteinuria.

4. The method of claim 1, wherein the formulation or product of manufacture is administered by inhalation, intravenously (IV), intradermally, intrathecally, sub- or intra-dermally, topically or intramuscularly (IM).

5. The method of any of 1, or any of the preceding claims, wherein the sunitinib (or SUTENT™), or salts or formulations thereof, is administered using, administering or implanting: a kit, an implant, a pump, a device, a subcutaneous infusion device, a continuous subcutaneous infusion device, an infusion pen, a needle, a reservoir, an ampoule, a vial, a syringe, a cartridge, a pen, a disposable pen or jet injector, a prefilled pen or a syringe or a cartridge, a cartridge or a disposable pen or jet injector, a two chambered or multi-chambered pump.

6-7 (canceled)

8. A method for inhibiting the activity of a cell comprising a ZAP70 kinase in vivo, in vitro or ex vivo, comprising contacting the cell with a formulation or product of manufacture comprising sunitinib, or salts or formulations thereof.

9. The method of claim 8, wherein the cell is a lymphocyte.

10. The method of claim 9, wherein the lymphocyte is a T cell, a B cell or a natural killer (NK) cell.

11. The method of claim 10, wherein the cell is a hyperactive T cell that mediates or initiates or aggravates a disease or condition.

12. The method of claim 11, wherein the disease or condition is selected from the group consisting of: an autoimmune disease, organ transplant rejection, graft-versus-host disease (GVHD) and B cell chronic lymphocytic leukemia (CLL).

13. The method of claim 12, wherein the autoimmune disease is or comprises: an allogeneic hematopoietic cell transplantation (HCT) in patients, rheumatoid arthritis (RA), inflammatory bowel disease (IBD), Crohn's disease, type 1 diabetes, multiple sclerosis, uncontrollable bullous pemphigoid, colitis, and proteinuria.

14. The method of claim 1, wherein the salts or formulations comrprise sunitinib malate.

15. The method of claim 1, wherein the formulation is formulated for administration in vivo; or as a pharmaceutical formulation or composition, or for enteral or parenteral administration, or as a tablet, pill, capsule, lozenge, gel, hydrogel, geltab, liquid, lotion, aerosol, patch, spray, or implant.

16. The method of claim 1, wherein the formulation is formulated as a liposome, a nanoparticle or a nanolipoparticle.

17. The method of claim 8, wherein the salts or formulations comrprise sunitinib malate.
